(19) United States
(12) Patent Application Publication
(10) Pub. No.: US 2026/0193228 A1
(43) Pub. Date: Jul. 9, 2026

(54) TRIAZINONE DERIVATIVES AS NLRP3 INHIBITORS

(71) Applicant: Hoffmann-La Roche Inc., Little Falls, NJ (US)

(72) Inventors: Thomas Alexander ALANINE, Nottingham (GB); Lea Aurelie BOUCHE, Basel (CH); Wolfgang GUBA, Müllheim (DE); Georg JAESCHKE, Basel (CH); Stefanie Katharina MESCH, Basel (CH); Jonathan Martin SHANNON, Nottingham (GB); Andreas Michael TOSSTORFF, Muenchenstein (CH)

(73) Assignee: Hoffmann-La Roche Inc., Little Falls, NJ (US)

(21) Appl. No.: 19/128,439

(22) PCT Filed: Nov. 7, 2023
(86) PCT No.: PCT/EP2023/080899

§ 371 Date: May 8, 2025

(30) Foreign Application Priority Data
Nov. 9, 2022 (EP) 22206280.4

(51) Int. Cl. C07D 401/12; A61K 31/53; C07D 405/14

(52) U.S. Cl. CPC C07D 401/12; A61K 31/53; C07D 405/14

(57) ABSTRACT

The invention relates to novel compounds having the general formula (I) wherein R¹, R², R³, R^{4a}, R^{4b}, and R⁵ are as described herein, composition including the compounds and methods of using the compounds.

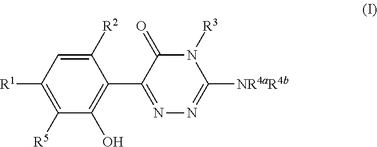

### FIELD OF THE INVENTION

The present invention relates to organic compounds useful for therapy and/or prophylaxis in a mammal, and in particular to compounds that modulate NLRP3 inhibition.

The present invention provides novel compounds of formula I

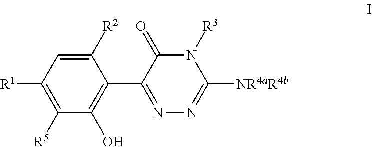

wherein
R¹ is H, acetyl, SF₅, halo, alkyl, alkoxy, haloalkyl, haloalkoxy or cyano;
R⁵ is H;
or R¹ and R⁵, and the atoms to which they are bonded, form either an 4-6 membered heterocycle ring comprising a single O heteroatom optionally substituted with one or two substituents independently selected from halo and alkyl, or R¹ and R⁵, and the atoms to which they are bonded, form a 4-6 membered cycloalkyl ring optionally substituted with 1 to 2 substituents independently selected from halo and alkyl;
R² is H, halo, alkyl, alkoxy, alkoxyalkyl, haloalkyl, or cycloalkyl, wherein cycloalkyl is optionally substituted with halo;
R³ is H or alkyl;
R^{4a} is a heterocycle ring optionally substituted with 1 to 3 substituents independently selected from halo, alkyl, haloalkyl, hydroxyalkyl, —OH, oxo, —CO₂H, cycloalkylalkyl or cycloalkyl optionally substituted with halo, and R^{4b} is H, or
R^{4a} and R^{4b}, and the N to which they are bonded, form a 9-member heterocycle comprising 1 or 2 N heteroatoms optionally substituted with 1 or 2 substituents independently selected from alkyl, —OH or halo;
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-la. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula I

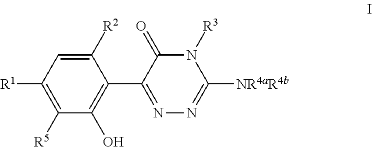

wherein
R¹ is H, acetyl, SF₅, halo, alkyl, alkoxy, haloalkyl, haloalkoxy or cyano;
R⁵ is H;
or R¹ and R⁵, and the atoms to which they are bonded, form either an 4-6 membered heterocycle ring comprising a single O heteroatom optionally substituted with one or two substituents independently selected from halo and alkyl, or R¹ and R⁵, and the atoms to which they are bonded, form a 4-6 membered cycloalkyl ring optionally substituted with 1 to 2 substituents independently selected from halo and alkyl;
R² is H, halo, alkyl, alkoxy, alkoxyalkyl, haloalkyl, cycloalkyl, wherein cycloalkyl is optionally substituted with halo;
R³ is H or alkyl;
R^{4a} is a heterocycle ring optionally substituted with 1 to 3 substituents independently selected from halo, alkyl, haloalkyl, hydroxyalkyl, —OH, oxo, —CO₂H, cycloalkylalkyl or cycloalkyl optionally substituted with halo, and R^{4b} is H, or
R^{4a} and R^{4b}, and the N to which they are bonded, form a 9-member heterocycle comprising 1 or 2 N heteroatoms optionally substituted with 1 or 2 substituents independently selected from alkyl, —OH or halo;
and pharmaceutically acceptable salts thereof.

The term “acetyl” denotes an —C(═O)CH₃ group.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl and cyclohexyl, and the like.

The term “cycloalkylalkyl” denotes an alkyl group wherein at least one of the hydrogen atoms of the alkyl group is replaced by a cycloalkyl group. Examples of cycloalkylalkyl include cyclopropylmethyl, cyclopropylethyl, cyclopropylbutyl, cyclobutylpropyl, 2-cyclopropylbutyl, cyclopentylbutyl, cyclohexylmethyl, and cyclohexylethyl.

The term “halogen”, “halide” and “halo” are used interchangeably herein and denote fluoro, chloro, bromo or iodo. Particular halogen is fluoro.

The term “haloalkyl” denotes a C₁₋₆-alkyl group wherein at least one of the hydrogen atoms of the C₁₋₆-alkyl group has been replaced by the same or different halogen atoms. Example of haloalkyl include fluoromethyl, difluoromethyl and trifluoromethyl. Particular example is trifluoromethyl.

The term “haloalkoxy” denotes a C₁₋₆-alkoxy group wherein at least one of the hydrogen atoms of the C₁₋₆-alkoxy group has been replaced by the same or different halogen atoms. Examples of haloalkoxy are difluoromethoxy, trifluoromethoxy, difluoroethoxy and trifluoroethoxy.

The term “heterocycle ring” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Examples for monocyclic saturated heterocycle rings are azetidinyl, diazepanyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, morpholinyl, and piperazinyl. Examples of polycyclic saturated heterocycle rings are azaspiroheptanyl, diazaspiroheptanyl, azaspirooctanyl, diazospirooctanyl, diazaspirononanyl, oxaazaspirooctanyl, and oxadiazaspirononanyl. One particular example of a polycyclic heterocycle ring includes hexahydro-pyrrolopyridinyl. Another particular example of a heterocycle ring is piperidinyl.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxypropyl, 2-hydroxy-1-propyl, 2-hydroxy-2-methyl-1-propyl, 3-hydroxy-1-propyl, and the like. Particular example of hydroxyalkyl is hydroxyethyl.

The term “nitrile” denotes a —C—N group.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is halo, haloalkyl or haloalkoxy and R^{S} is H; or R¹ and R⁵, and the atoms to which they are bonded, form either an 4-6 membered heterocycle ring comprising a single O heteroatom optionally substituted with one or two substituents independently selected from halo and alkyl, or R¹ and R⁵, and the atoms to which they are bonded, form a 4-6 membered cycloalkyl ring optionally substituted with 1 to 2 substituents independently selected from halo and alkyl.

An embodiment of the present invention provides compounds according to formula I as described herein, wherein R¹ is halo, haloalkyl or haloalkoxy and R⁵ is H; or R¹ and R⁵ and the atoms to which they are bonded, form either an 5 membered heterocycle ring comprising a single O heteroatom, or R¹ and R⁵, and the atoms to which they are bonded, form a 5 membered cycloalkyl ring.

An embodiment of the present invention provides compounds according to formula I as described herein, wherein R¹ is halo, haloalkyl or haloalkoxy.

An embodiment of the present invention provides compounds according to formula I as described herein, wherein R¹ is haloalkyl.

An embodiment of the present invention provides compounds according to formula I as described herein, wherein R⁵ is H, or R¹ and R⁵, and the atoms to which they are bonded, form either a 5 membered heterocycle ring comprising a single O heteroatom, or R¹ and R⁵, and the atoms to which they are bonded, form a 5 membered cycloalkyl ring.

An embodiment of the present invention provides compounds according to formula I as described herein, wherein R⁵ is H.

An embodiment of the present invention provides compounds according to formula I as described herein, wherein R² is H or alkyl.

An embodiment of the present invention provides compounds according to formula I as described herein, wherein R² is alkyl.

An embodiment of the present invention provides compounds according to formula I as described herein, wherein R³ is alkyl.

An embodiment of the present invention provides compounds according to formula I as described herein, wherein R^{4a} is a 6-membered heterocycle ring substituted with alkyl, hydroxyalkyl, cycloalkylalkyl or cycloalkyl optionally substituted with halo, and R^{4b} is H, or R^{4a} and R^{4b}, and the N to which they are bonded, form a 9-member heterocycle comprising 1 or 2 N heteroatoms optionally substituted with alkyl or halo.

An embodiment of the present invention provides compounds according to formula I as described herein, wherein R^{4a} is a 6-membered heterocycle ring substituted with alkyl, cycloalkylalkyl or cycloalkyl optionally substituted with halo, and R^{4b} is H, or R^{4a} and R^{4b}, and the N to which they are bonded, form a 9-member heterocycle comprising 1 or 2 N heteroatoms optionally substituted with alkyl or halo.

An embodiment of the present invention provides compounds according to formula I as described herein, wherein R^{4a} is a 6-membered heterocycle ring comprising a single N heteroatom substituted with alkyl or hydroxyalkyl, and R^{4b} is H, or R^{4a} and R^{4b}, and the N to which they are bonded, form a 9-member heterocycle comprising 1 or 2 N heteroatoms optionally substituted with alkyl.

An embodiment of the present invention provides compounds according to formula I as described herein, wherein R^{4a} is a 6-membered heterocycle ring comprising a single N heteroatom substituted with alkyl, and R^{4b} is H, or R^{4a} and R^{4b}, and the N to which they are bonded, form a 9-member heterocycle comprising 1 or 2 N heteroatoms optionally substituted with alkyl.

An embodiment of the present invention provides compounds according to formula I as described herein, wherein R^{4a} is a 6-membered heterocycle ring comprising a single N heteroatom substituted with alkyl, and R^{4b} is H, or R^{4a} and R^{4b}, and the N to which they are bonded, form a 9-member heterocycle comprising 2 N heteroatoms wherein the 9-member heterocycle comprising 2 N heteroatoms is substituted with alkyl.

An embodiment of the present invention provides compounds according to formula I as described herein, wherein R^{4a} is ethylpiperidyl and R^{4b} is H.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H, halo, alkyl, haloalkyl, cycloalkyl, wherein cycloalkyl is optionally substituted with halo;
R³ is alkyl;
R^{4a} is a 6-membered heterocycle ring substitute with alkyl, hydroxyalkyl, cycloalkylalkyl or cycloalkyl optionally substituted with halo, and R^{4b} is H, or
R^{4a} and R^{4b}, and the N to which they are bonded, form a 9-member heterocycle comprising 1 or 2 N heteroatoms optionally substituted with alkyl or halo; and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H, halo, alkyl, haloalkyl, cycloalkyl, wherein cycloalkyl is optionally substituted with halo;
R³ is alkyl;
R^{4a} is a 6-membered heterocycle ring substitute with alkyl, cycloalkylalkyl or cycloalkyl optionally substituted with halo, and R^{4b} is H, or
R^{4a} and R^{4b}, and the N to which they are bonded, form a 9-member heterocycle comprising 1 or 2 N heteroatoms optionally substituted with alkyl or halo; and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H or alkyl;
R³ is alkyl;
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-member heterocycle comprising 1 or 2 N heteroatoms optionally substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H or alkyl;
R³ is alkyl;
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-member heterocycle comprising 1 or 2 N heteroatoms optionally substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is alkyl;
R³ is alkyl;
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-member heterocycle comprising 1 or 2 N heteroatoms optionally substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H or alkyl;
R³ is alkyl;
R^{4a} is a 6-membered heterocycle ring comprising a single N heteroatom substituted with alkyl, and R^{4b} is H, or
R^{4a} and R^{4b}, and the N to which they are bonded, form a 9-member heterocycle comprising 1 or 2 N heteroatoms optionally substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is alkyl;
R³ is alkyl;
R^{4a} is a 6-membered heterocycle ring comprising a single N heteroatom substituted with alkyl, and R^{4b} is H, or
R^{4a} and R^{4b}, and the N to which they are bonded, form a 9-member heterocycle comprising 1 or 2 N heteroatoms optionally substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H or alkyl;
R³ is alkyl;
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-member heterocycle comprising 2 N heteroatoms wherein the 9-member heterocycle comprising 2 N heteroatoms is substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is alkyl;
R³ is alkyl;
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-member heterocycle comprising 2 N heteroatoms wherein the 9-member heterocycle comprising 2 N heteroatoms is substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is H or alkyl;
R³ is alkyl;
R^{4a} is a 6-membered heterocycle ring comprising a single N heteroatom substituted with alkyl, and R^{4b} is H, or
R^{4a} and R^{4b}, and the N to which they are bonded, form a 9-member heterocycle comprising 2 N heteroatoms wherein the 9-member heterocycle comprising 2 N heteroatoms is substituted with alkyl;
and pharmaceutically acceptable salts thereof.

An embodiment of the present invention provides compounds according to formula I as described herein, wherein
R¹ is halo, haloalkyl, or haloalkoxy;
R⁵ is H;
or R¹ and R⁵, and the atoms to which they are bonded, form either an 5 membered heterocycle ring comprising a single O heteroatom, or R¹ and R⁵, and the atoms to which they are bonded, form a 5 membered cycloalkyl ring;
R² is alkyl;
R³ is alkyl;
R^{4a} is a 6-membered heterocycle ring comprising a single N heteroatom substituted with alkyl, and R^{4b} is H, or
R^{4a} and R^{4b}, and the N to which they are bonded, form a 9-member heterocycle comprising 2 N heteroatoms wherein the 9-member heterocycle comprising 2 N heteroatoms is substituted with alkyl;
and pharmaceutically acceptable salts thereof.

An embodiment of the present invention provides compounds according to formula I as described herein, wherein
R¹ is haloalkyl;
R⁵ is H;
R² is H or alkyl;
R³ is alkyl;
R^{4a} is ethylpiperidyl and R^{4b} is H;
and pharmaceutically acceptable salts thereof.

An embodiment of the present invention provides compounds according to formula I as described herein, wherein
R¹ is haloalkyl;
R⁵ is H;
R² is alkyl;
R³ is alkyl;
R^{4a} is ethylpiperidyl and R^{4b} is H;
and pharmaceutically acceptable salts thereof.

Particular examples of compounds of formula I as described herein are selected from
3-[[(3R)-1-Ethyl-3-piperidyl]amino]-6-[2-hydroxy-4-(trifluoromethyl)phenyl]-4-methyl-1,2,4-triazin-5-one;
3-[[(3R)-1-Ethyl-3-piperidyl]amino]-6-[2-hydroxy-6-methyl-4-(trifluoromethyl)phenyl]-4-methyl-1,2,4-triazin-5-one;
and pharmaceutically acceptable salts thereof.

Other particular examples of compounds of formula I as described herein are selected from
3-[[(3R)-1-Ethyl-3-piperidyl]amino]-6-(4-hydroxy-6-methyl-indan-5-yl)-4-methyl-1,2,4-triazin-5-one;
3-[[(3R)-1-(2-Hydroxyethyl)-3-piperidyl]amino]-6-(4-hydroxy-6-methyl-indan-5-yl)-4-methyl-1,2,4-triazin-5-one;
3-[[(3R)-1-Ethyl-3-piperidyl]amino]-6-(4-hydroxy-6-methyl-2,3-dihydrobenzofuran-5-yl)-4-methyl-1,2,4-triazin-5-one;
6-(4-Chloro-2-hydroxy-6-methyl-phenyl)-3-[[(3R)-1-ethyl-3-piperidyl]amino]-4-methyl-1,2,4-triazin-5-one;
and pharmaceutically acceptable salts thereof.

A preferred example of compounds of formula I as described herein is 3-[[(3R)-1-ethyl-3-piperidyl]amino]-6-[2-hydroxy-6-methyl-4-(trifluoromethyl)phenyl]-4-methyl-1,2,4-triazin-5-one and pharmaceutically acceptable salts thereof.

Other preferred examples of compounds of formula I as described herein are selected from
3-[[(3R)-1-Ethyl-3-piperidyl]amino]-6-(4-hydroxy-6-methyl-indan-5-yl)-4-methyl-1,2,4-triazin-5-one;
3-[[(3R)-1-Ethyl-3-piperidyl]amino]-6-(4-hydroxy-6-methyl-2,3-dihydrobenzofuran-5-yl)-4-methyl-1,2,4-triazin-5-one;
and pharmaceutically acceptable salts thereof.

Processes for the manufacture of compounds of formula I as described herein are an object of the invention.

The present compounds of formula I and their pharmaceutically acceptable salts can be prepared by methods known in the art, for example, by processes described below, which process comprises reacting a compound of formula III to provide a compound of formula I through a protecting group (PG) cleavage. Preferably, the protecting group is a methyl ether. Preferably, the methyl ether is cleaved with boron tribromide (BBr₃) in dichloromethane.

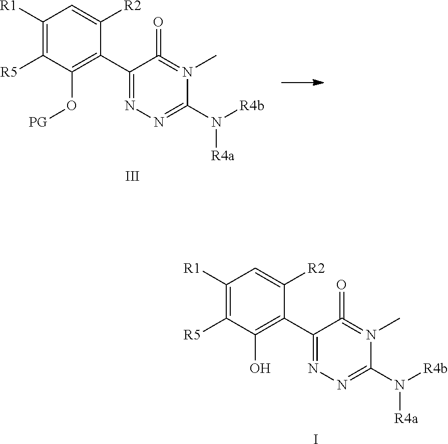

### General Synthetic Schemes

The compounds of general formula I can be prepared with the process variant described below following to the scheme 1. The starting material is commercially available or may be prepared in accordance with known methods.

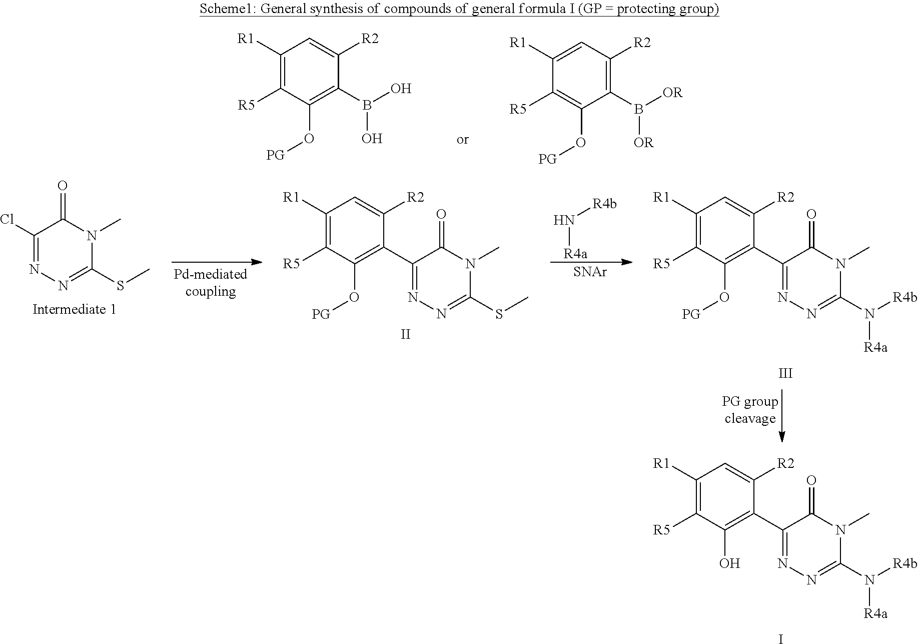

Compounds of general formula II were obtained using a Palladium-mediated Suzuki-Miyaura cross-coupling in the presence of intermediate 1 and a boronic acid or ester with a general formula as depicted in scheme 1 in conditions well known to the skilled person with. Subsequently a nucleophilic aromatic substitution (SNAr) in order to prepare compounds of general formula III was carried out with suitable secondary amines of general formula HNR^{4b}R^{4a} wherein R^{4a} and R^{4b} have the meaning given in the claims of this current invention in the presence of pyridine under heating. Alternatively, the reaction can be also done under microwave irradiation. Finally, a last step of protecting group cleavage was needed. In the case of a methyl ether-protecting group, this is usually cleaved with boron tribromide (BBr₃) in dichloromethane delivering the compounds of general formula I.

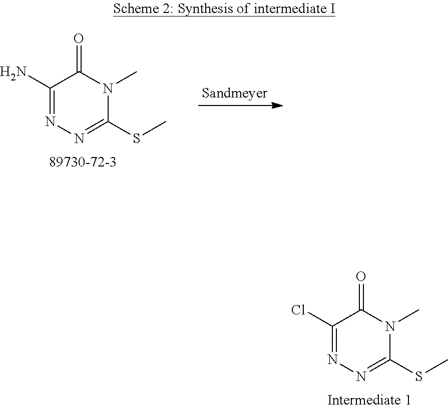

Intermediate 1 was synthesized as described in the experimental section via a Sandmeyer type reaction using commercially available 6-amino-4-methyl-3-methylsulfanyl-1,2,4-triazin-5-one (CAS #89730-72-3) in the presence of copper (I) chloride and lithium chloride in Acetonitrile at 60° C.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to formula I as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use of a compound according to formula I as described herein in the treatment or prophylaxis of a disease, disorder or condition selected from Alzheimer's disease and Parkinson's disease.

An embodiment of the present invention is the use a compound according to formula I as described herein for use in the treatment or prophylaxis of a disease, disorder or condition selected from Asthma or COPD.

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Asthma or COPD.

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for the preparation of a medicament for the treatment or prophylaxis of a disease, disorder or condition selected from Asthma or COPD.

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Asthma or COPD, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (~10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 g/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

hERG Screening Assay

In the drug development process of small molecules, one of the most frequent adverse side effects, leading to the failure of drugs, is the cardiac arrhythmias. Such failure is often related to the capacity of the drug to inhibit the human ether-a-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂ 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

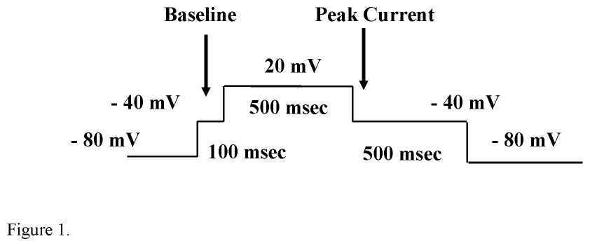

Cells were held at a resting voltage of −80 mV and they were stimulated by a voltage pattern shown in FIG. 1 (pulse pattern used to elicit outward K+ current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

| | |
|---|---|
| | $I ( C ) = \frac{100}{1 + {( C / IC 50 )}^{h}}$ |
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter Logistic Model (fit=(A+(B/(1+((x/C)\hat{ }D)))), where A=0 and B=100).

The results of the hERG assay are summarised in Table 2 below as hERG IC₂₀.

### Transcellular P-gp Assay:

The general assay uses transfected LLC-PK1 cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO2 incubator with an orbital shaker. After the addition of a test compound at e.g. 1 μM to the wells (1×105 cells/well), the 96-well hepatocyte suspension culture plates are incubated in a 5% CO2 at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

TABLE 1
NLRP3 inhibitory activity

| | THP-1 pyroptosis | Human whole blood IL-1β |
|---|---|---|
| | Assay | Assay IC₅₀ |
| Example No. | IC₅₀ (nM) | (nM) |
| 1 | 615.7 | |
| 2 | 31.3 | 52 |
| 3 | 17 | 41 |
| 4 | 248 | |
| 5 | 39 | 33 |
| 6 | 33 | 41 |

TABLE 2
hERG inhibition assay

| | hERG assay |
|---|---|
| Example No. | IC₂₀ (μM) |
| 2 | >10 |
| 3 | >10 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### Experimental Methods

### Abbreviations

| | |
|---|---|
| ACN | acetonitrile |
| CH₂Cl₂ | dichloromethame |
| DIPEA | diisopropylethylamine |
| DMF | N,N-dimethylformamide |
| DMSO | Dimethyl sulfoxide |
| EtOAc | Ethyl acetate |
| h, hrs | hour, hours |
| HPLC | High-performance liquid chromatography |
| min(s) | Minute(s) |
| MSD | mass selective detector |
| NMR | Nuclear magnetic resonance spectroscopy |
| NMP | N-methyl-2-pyrrolidinone |
| prep | preparative |
| PE | Petrol ether |
| RP | Reverse phase |
| TFA | Trifluoroacetic acid |
| TLC | Thin-layer chromatography |

### EXAMPLES

All examples and intermediates were prepared under nitrogen atmosphere if not specified otherwise.

### Synthesis of Intermediates:

### Intermediate 1

### 6-Chloro-4-methyl-3-methylsulfanyl-1,2,4-triazin-5-one

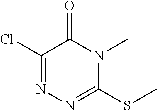

Isoamyl nitrite (2.02 mL, 15.01 mmol, 2.02 eq) was added dropwise to a mixture of 6-amino-4-methyl-3-methylsulfanyl-1,2,4-triazin-5-one (CAS #89730-72-3, 1.28 g, 7.43 mmol, 1.0 eq), benzyltriethylammonium chloride (2.54 g, 11.2 mmol, 1.5 eq), copper (I) chloride (1.47 g, 14.9 mmol, 2.0 eq) and lithium chloride (472.6 mg, 11.2 mmol, 1.5 eq) in MeCN (30 mL) heated at 60° C. for 2 h. The reaction mixture was cooled, diluted with TBME (50 ml) and filtered through celite. The filtrate was washed with water (50 ml), dried (MgSO4), filtered and concentrated in vacuo. The residue was purified by chromatography on silica gel (40 g cartridge, 0-50% EtOAc/isohexane) to afford the title compound (570 mg, 39%) as a white solid. LCMS: m/z 192.3/194.0 [M+H]⁺, ESI pos.

### Syntheses of the Examples

### Example 1

### 3-[[(3R)-1-Ethyl-3-piperidyl]amino]-6-[2-hydroxy-4-(trifluoromethyl)phenyl]-4-methyl-1,2,4-triazin-5-one; Formic Acid

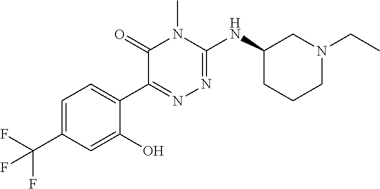 [SMILES: CCN1CCC[C@@H](Nc2nnc(-c3ccc(C(F)(F)F)cc3O)c(=O)n2C)C1]

### Step A: 6-[2-Methoxy-4-(trifluoromethyl)phenyl]-4-methyl-3-methylsulfanyl-1,2,4-triazin-5-one

To a stirred solution of 6-chloro-4-methyl-3-methylsulfanyl-1,2,4-triazin-5-one Intermediate 1 (500 mg, 2.48 mmol, 1.0 eq), 2-methoxy-4-(trifluoromethyl)-phenylboronic acid (670 mg, 3.02 mmol, 1.22 eq) and Xphos Pd G3 (215 mg, 0.25 mmol, 0.1 eq) in 1,4-Dioxane (5 mL) was added sodium carbonate (aq saturated) (0.5 mL, 7.44 mmol, 3.0 eq). The mixture was degassed with N₂ three times, and the reaction mixture was stirred at 100° C. for 6 h then left standing at r.t. overnight. The reaction mixture was diluted with water (15 mL) and EtOAc (40 mL). The layers were separated, and the aqueous layer was extracted with EtOAc (2×15 mL). The combined organic was dried (Na₂SO₄), filtered, and concentrated to dryness. The crude was purified by flash column chromatography on silica gel (24 g cartridge, 0-100% TBME/isohexane) to afford the title compound (411 mg, 45%) as a light-yellow solid. LCMS m/z 332.0 [M+H]⁺ ESI pos.

### Step B: 3-[[(3R)-1-Ethyl-3-piperidyl]amino]-6-[2-methoxy-4-(trifluoromethyl)phenyl]-4-methyl-1,2,4-triazin-5-one

A solution of 6-[2-methoxy-4-(trifluoromethyl)phenyl]-4-methyl-3-methylsulfanyl-1,2,4-triazin-5-one (step A) (278 mg, 0.76 mmol, 1.0 eq) and (3R)-1-ethylpiperidin-3-amine (610 mg, 4.76 mmol, 6.3 eq) in Pyridine (2 mL) was heated at 90° C. for 72 h. The reaction mixture was cooled and diluted with DCM (50 mL) and water (10 mL). The layers were separated and the organic layer was washed with brine (3×10 mL), dried (Na₂SO₄), filtered and concentrated to dryness. The crude was then purified by flash column chromatography on silica gel (24 g cartridge, DCM load, 0-10% MeOH (0.7 M NH₃)/DCM) to afford the title compound (212 mg, 61%) as a thick brown oil. LCMS m/z 412.2 [M+H]⁺ ESI pos.

### Step C: 3-[[(3R)-1-ethyl-3-piperidyl]amino]-6-[2-hydroxy-4-(trifluoromethyl)phenyl]-4-methyl-1,2,4-triazin-5-one; formic acid

A solution of boron tribromide (2.9 mL, 2.90 mmol, 1 M in DCM, 5.01 eq) was added dropwise to 3-[[(3R)-1-ethyl-3-piperidyl]amino]-6-[2-methoxy-4-(trifluoromethyl)phenyl]-4-methyl-1,2,4-triazin-5-one (step B) (238 mg, 0.58 mmol, 1.0 eq) in DCM (8 mL) at 0° C. The reaction mixture was stirred for 30 min and then quenched with 0.7 M NH₃ MeOH (10 mL) and stirred for 30 min at r.t. The reaction mixture was concentrated under reduced pressure. The crude product was dissolved in DMSO (8 mL), filtered, and purified by reversed phase preparative HPLC (Gilson) using a Phenomenex Gemini NC—C₁₈ prep column, 110 Å, 5 μm, 30 mm×150 mm, flow rate 42 mL min-1 eluting with a 0.1% Formic Acid in water-MeCN gradient over 15 min. At-column dilution pump gives 5 mL min⁻¹ MeCN for 1.2 min. Gradient information: 0.0-1.0 min, 90% MeCN; 1.0-11.0 min, ramped from 90% MeCN to 65% MeCN; 11.1 to 14.0 min, ramped from 65% MeCN to 100% MeCN; 14.1-15.0 min, equilibrated from 100% to 90% MeCN. The clean fractions were evaporated in a Genevac to afford the title compound (142 mg, 54% yield) as a yellow solid. LCMS m/z 398.4 [M+H]⁺ ESI pos.

### Example 2

### 3-[[(3R)-1-ethyl-3-piperidyl]amino]-6-[2-hydroxy-6-methyl-4-(trifluoromethyl)phenyl]-4-methyl-1,2,4-triazin-5-one

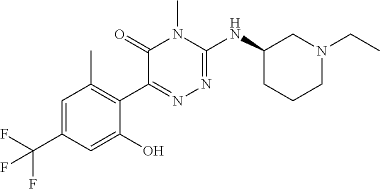 [SMILES: CCN1CCC[C@@H](Nc2nnc(-c3c(C)cc(C(F)(F)F)cc3O)c(=O)n2C)C1]

### Step A: 6-[2-Methoxy-6-methyl-4-(trifluoromethyl)phenyl]-4-methyl-3-methylsulfanyl-1,2,4-triazin-5-one

To a stirred solution of 6-chloro-4-methyl-3-methylsulfanyl-1,2,4-triazin-5-one Intermediate 1 (300 mg, 1.49 mmol, 1.0 eq), [2-methoxy-6-methyl-4-(trifluoromethyl)phenyl]boronic acid (423.4 mg, 1.81 mmol, 1.22 eq) and Xphos Pd G3 (129.0 mg, 0.15 mmol, 0.1 eq) in 1,4-Dioxane (3 mL) was added sodium carbonate (aq saturated) (0.3 mL) was added. The mixture was degassed with N₂ three times and stirred at 90° C. 12 h under N₂. Water (15 mL) and EtOAc (40 mL) were added. The layers were separated, and the aqueous layer was extracted with EtOAc (2×15 mL). The combined organic was dried (Na₂SO₄), filtered and concentrated to dryness. The product was purified by flash column chromatography on silica gel (24 g cartridge, 0-60% TBME/isohexane) to afford the title compound (87.0 mg, 16%) as a light-yellow gum.

LCMS m/z 346.23 [M+H]⁺ ESI pos.

### Step B: 3-[[(3R)-1-ethyl-3-piperidyl]amino]-6-[2-hydroxy-6-methyl-4-(trifluoromethyl)phenyl]-4-methyl-1,2,4-triazin-5-one

A solution of 6-[2-methoxy-6-methyl-4-(trifluoromethyl)phenyl]-4-methyl-3-methylsulfanyl-1,2,4-triazin-5-one (80.0 mg, 0.23 mmol, 1.0 eq) and (3R)-1-ethylpiperidin-3-amine;dihydrochloride (293.5 mg, 1.46 mmol, 6.3 eq) in Pyridine (2 mL) was heated at 90° C. for 4 d. The reaction mixture was cooled to r.t., DCM (50 mL) and water (10 mL) were added. The layers were separated, and the organic layer was washed with brine (3×10 mL), dried over Na₂SO₄, filtered and concentrated to dryness. The crude intermediate was then purified by flash column chromatography on silica gel (24 g cartridge, 0-10% MeOH (0.7 M NH₃)/DCM to afford as a brown solid (21.0 mg). The brown solid was dissolved in DCM (2 mL) cooled to 0° C. and boron tribromide (1 M in DCM) (1.16 mL, 1.16 mmol, 5.0 eq) was added and then allowed to warm to rt and stirred for 18 h. The reaction mixture was added to a stirred solution of 0.7M NH3 in MeOH (10 mL) at 0° C., the reaction mixture was allowed to warm to rt overnight and then concentrated. The brown residue was dissolved in DCM (25 mL) and water (10 mL), layers were separated, and the organic layer was dried (MgSO₄) and concentrated to dryness. The product was purified by chromatography on silica gel (12 g cartridge, 0-10% MeOH (0.7 M NH₃)/DCM) to afford the title compound (7.0 mg, 7%) as a light brown solid. LCMS m/z 412.24 [M+H]⁺ ESI pos.

### Intermediate 2

### 6-Iodo-4-methyl-3-methylsulfanyl-1,2,4-triazin-5-one

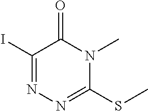

Isoamylnitrite (320.0 μL, 2.38 mmol, 2.05 eq) was added dropwise to a mixture of 6-amino-4-methyl-3-methylsulfanyl-1,2,4-triazin-5-one (CAS #89730-72-3; 200.0 mg, 1.16 mmol, 1.0 eq), tetrabutylammonium iodide (648.0 mg, 1.75 mmol, 1.51 eq), copper iodide (447.0 mg, 2.35 mmol, 2.02 eq) and iodolithium (330.0 mg, 2.47 mmol, 2.12 eq) in MeCN (4 mL) heated at 60° C. for 3 h. The reaction mixture was cooled, diluted with EtOAc (50 mL) and filtered through celite, rinsing with EtOAc (2×50 mL). The filtrate was diluted with water (100 mL) and the separated aqueous layer was further extracted with EtOAc (2×50 mL). The combined organic layers were dried (Na₂SO₄), filtered and evaporated to give the crude product. The residue was purified by chromatography on silica gel (40 g cartridge, 0-100% EtOAc/isohexane) to afford the title compound (156.0 mg, 46% yield) as a yellow solid. LCMS: m/z 284.0 [M+H]⁺ ESI pos.

### Intermediate 3:

### 2-(4-Benzyloxy-6-methyl-indan-5-yl)-4,4,5,5-tetramethyl-1,3,2-dioxaborolane

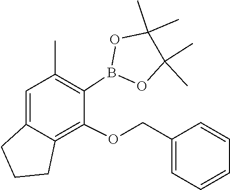 [SMILES: Cc1cc2c(c(OCc3ccccc3)c1B1OC(C)(C)C(C)(C)O1)CCC2]

### Step A: 5-Bromo-6-methyl-indan-4-ol

Sodium nitrite (200.0 mg, 2.9 mmol, 1.3 eq) in water (5 mL) was added dropwise to a stirred solution of 5-bromo-6-methyl-indan-4-amine (500.0 mg, 2.21 mmol, 1.0 eq, CAS #2411531-03-6) in sulfuric acid (10.0 mL, 20.0 mmol, 9.0 eq) (2 M in THF) at 0° C. The reaction was stirred for ~30 mins, then was allowed to return to rt over ~30 mins. The reaction mixture was then added dropwise to a stirred solution of sulfuric acid (10.0 mL, 20.0 mmol, 9.0 eq) (2 M in THF) at 50° C. and the reaction was stirred for a further 2.5 h. The reaction was allowed to cool to rt, then was diluted with water (100 mL) and EtOAc (100 mL). The layers were separated and the aqueous phase was extracted again with EtOAc (2×50 mL). The combined organic layers were washed with brine (1×50 mL), dried with MgSO₄ and concentrated in vacuo. The crude material was purified by column chromatography on silica gel (0-20% EtOAc/isohexane) to give the title compound (211.3 mg, 42% yield) as an off white solid. ¹H NMR (500 MHz, DMSO) δ 9.13 (br s, 1H), 6.70 (s, 1H), 2.80 (t, 2H), 2.76 (t, 2H), 2.27 (s, 3H), 1.97 (p, 2H).

### Step B: 4-Benzyloxy-5-bromo-6-methyl-indane

Potassium carbonate (185.0 mg, 1.34 mmol, 2.0 eq) was added to a stirred solution of 5-bromo-6-methyl-indan-4-ol (150.0 mg, 0.66 mmol, 1.0 eq) in acetone (5 mL) and the reaction was stirred at rt for 5 mins. Benzyl bromide (0.1 mL, 0.84 mmol, 1.27 eq) was then added dropwise and the reaction was stirred for a further ~36 h. The reaction mixture was concentrated in vacuo, then was taken up in DCM (30 mL) and water (30 mL). The organic layer was separated and the aqueous was extracted again with DCM (2×30 mL). The combined organic layers were washed with brine (1×30 mL), dried with MgSO₄ and concentrated in vacuo. The crude material was purified by column chromatography on silica gel (0-10% EtOAc/isohexane) to give the title compound (191.3 mg, 90% yield) as a light yellow oil. ¹H NMR (500 MHz, DMSO) δ 7.54-7.48 (m, 2H), 7.44-7.38 (m, 2H), 7.38-7.32 (m, 1H), 7.01 (s, 1H), 4.95 (s, 2H), 2.89 (t, 2H), 2.80 (t, 2H), 2.33 (s, 3H), 2.06-1.95 (m, 2H).

### Step C: 2-(4-Benzyloxy-6-methyl-indan-5-yl)-4,4,5,5-tetramethyl-1,3,2-dioxaborolane

n-Butyl lithium (1.6 M in hexanes) (4.41 mL, 7.06 mmol, 1.6 eq) was added dropwise to a stirred solution of 4-benzyloxy-5-bromo-6-methyl-indane (1.4 g, 4.41 mmol, 1.0 eq) and 2-isopropoxy-4,4,5,5-tetramethyl-1,3,2-dioxaborolane (1.35 mL, 6.62 mmol, 1.5 eq) in THF (20 mL) at −78° C. and the reaction was stirred for 3 hours. The reaction was quenched by the slow addition of sat. aq. NH₄Cl (20 mL). The mixture was allowed to warm to rt over 18 hours. The layers were separated and the aqueous layer extracted with EtOAc (2×20 mL) after which the combined organics were concentrated in vacuo. The crude product was purified by silica gel chromatography (0-5% (heptane/EtOAc)) to give the title compound (975.0 mg, 60% yield) as a light brown solid. ¹H NMR (500 MHz, DMSO) δ 7.48-7.42 (m, 2H), 7.41-7.35 (m, 2H), 7.35-7.30 (m, 1H), 6.81 (s, 1H), 4.91 (s, 2H), 2.83 (dt, 4H), 2.25 (s, 3H), 1.97 (p, 2H), 1.22 (s, 12H).

### Example 3

### 3-[[(3R)-1-Ethyl-3-piperidyl]amino]-6-(4-hydroxy-6-methyl-indan-5-yl)-4-methyl-1,2,4-triazin-5-one

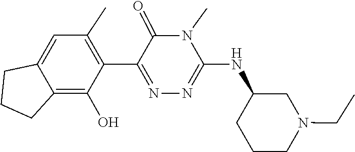 [SMILES: CCN1CCC[C@@H](Nc2nnc(-c3c(C)cc4c(c3O)CCC4)c(=O)n2C)C1]

### Step A: 6-(4-Benzyloxy-6-methyl-indan-5-yl)-4-methyl-3-methylsulfanyl-1,2,4-triazin-5-one

A mixture of 2-(4-benzyloxy-6-methyl-indan-5-yl)-4,4,5,5-tetramethyl-1,3,2-dioxaborolane Intermediate 3 (322.0 mg, 0.88 mmol, 1.11 eq), 6-iodo-4-methyl-3-methylsulfanyl-1,2,4-triazin-5-one Intermediate 2 (225.0 mg, 0.79 mmol, 1.0 eq), K₂CO₃ (352.0 mg, 2.55 mmol, 3.2 eq) and Pd-170 (111.0 mg, 0.16 mmol, 0.21 eq) in MeCN (12 mL) and water (4 mL) were degassed with N₂ for 5 mins, then was heated to 60° C. for 21 h. A slow stream of N₂ was added via a needle to the reaction mixture to try limit formation of diMeS side-products. The reaction was cooled, concentrated and loaded onto Celite. The crude product was purified by column chromatography on C18 silica gel (40 g cartridge, 10-100% [0.1% formic acid MeCN: 0.1% Aq. formic acid], to afford the title compound (86.0 mg, 24% yield) as a yellow solid. LCMS m/z 394.2 [M+H]⁺ ESI pos.

### Step B: 6-(4-Benzyloxy-6-methyl-indan-5-yl)-4-methyl-3-[[(3R)-1-benzyl-3-piperidyl]amino]-1,2,4-triazin-5-one; Formic Acid Salt

6-(4-Benzyloxy-6-methyl-indan-5-yl)-4-methyl-3-methylsulfanyl-1,2,4-triazin-5-one (70.0 mg, 0.18 mmol, 1.0 eq) was added to a small MW vial and (R)-3-amino-1-benzylpiperidine (170.0 mg, 0.89 mmol, 5.02 eq; CAS #168466-84-0) was added. The mixture was then irradiated at 180° C. for 2 h. The reaction was cooled to rt, the resulting thick black residue dissolved with DCM and loaded onto Celite. The crude product was purified by reverse-phase column chromatography on C18 silica gel (26 g cartridge, 10-100% [0.1% formic acid MeCN: 0.1% aq formic acid], to afford the title compound (35.0 mg, 36% yield) as a light brown solid, and a further portion of title compound (18.0 mg, 17% yield) as a dark brown solid. LCMS m/z 536.4 [M+H]⁺ ESI pos.

### Step C: 3-[[(3R)-1-Ethyl-3-piperidyl]amino]-6-(4-hydroxy-6-methyl-indan-5-yl)-4-methyl-1,2,4-triazin-5-one

Pd/C (Type 39) (32.0 mg, 0.02 mmol, 0.25 eq) and Pd/C (Type 87) (16.0 mg, 0.02 mmol, 0.25 eq) were added to a stirred solution of 6-(4-benzyloxy-6-methyl-indan-5-yl)-4-methyl-3-[[(3R)-1-benzyl-3-piperidyl]amino]-1,2,4-triazin-5-one; formic acid salt (35.0 mg, 0.1 mmol, 1.0 eq) in acetonitrile (2.1 mL). The hydrogenation vessel was placed under an atmosphere of hydrogen gas (2 bar) at rt and vigorously stirred for 4 h. The reaction was filtered through a plug of celite, rinsing with EtOH, and concentrated to dryness to give the crude product. The crude product was purified by column chromatography on silica gel (24 g cartridge, 0-10% MeOH (0.7M NH₃)/DCM), to give the title compound (20.0 mg, 85% yield), after freeze-drying, as a light yellow freeze-dried solid. LCMS m/z 384.3 [M+H]⁺ ESI pos.

### Example 4

### 3-[[(3R)-1-(2-Hydroxyethyl)-3-piperidyl]amino]-6-(4-hydroxy-6-methyl-indan-5-yl)-4-methyl-1,2,4-triazin-5-one

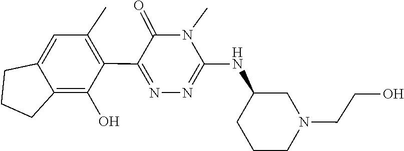 [SMILES: Cc1cc2c(c(O)c1-c1nnc(N[C@@H]3CCCN(CCO)C3)n(C)c1=O)CCC2]

### Step A: 6-(4-Hydroxy-6-methyl-indan-5-yl)-4-methyl-3-[[(3R)-3-piperidyl]amino]-1,2,4-triazin-5-one

Pd/C (Type 39) (20.0 mg, 0.01 mmol, 0.29 eq) and Pd/C (Type 87) (10.0 mg, 0.01 mmol, 0.29 eq) were added to a stirred solution of 6-(4-benzyloxy-6-methyl-indan-5-yl)-4-methyl-3-[[(3R)-1-benzyl-3-piperidyl]amino]-1,2,4-triazin-5-one; formic acid salt Example 3 Step B (19.0 mg, 0.03 mmol, 1.0 eq) in 1,4-dioxane (3 mL). The hydrogenation vessel was placed under an atmosphere of hydrogen gas (2 bar) at rt and vigorously stirred for 18 h. Additional Pd/C (Type 39) (20.0 mg, 0.01 mmol, 0.29 eq) and Pd/C (Type 87) (10.0 mg, 0.01 mmol, 0.29 eq) were added and the reaction was subjected to the conditions again for 24 h. The reaction was filtered through a plug of Celite, rinsing with EtOH, and concentrated to dryness to give the title compound (7 mg, 42% yield), which was used in the next step without further purification. LCMS m/z 356.3 [M+H]⁺ ESI pos.

### Step B: 3-[[(3R)-1-(2-Hydroxyethyl)-3-piperidyl]amino]-6-(4-hydroxy-6-methyl-indan-5-yl)-4-methyl-1,2,4-triazin-5-one

2-Iodoethanol (3.39 mg, 0.02 mmol, 1.0 eq) in DMF (0.100 mL) was added dropwise to a stirred solution of 6-(4-hydroxy-6-methyl-indan-5-yl)-4-methyl-3-[[(3R)-3-piperidyl]amino]-1,2,4-triazin-5-one (7.0 mg, 0.02 mmol, 1.0 eq) and DIPEA (5.15 uL, 0.03 mmol, 1.5 eq) in DMF (0.30 mL) at rt, and the reaction mixture was stirred for 3 days. The reaction mixture was concentrated to dryness then loaded onto Celite. The crude was then purified by reverse-phase flash column chromatography (C18 13 g cartridge, 10-100% MeCN/aq. 10 mM NH₄HCO₃) to afford the title compound (2.0 mg, 24% yield) after freeze-drying, as an off-white solid. LCMS m/z 400.5 [M+H]⁺ ESI pos.

### Example 5

### 3-[[(3R)-1-Ethyl-3-piperidyl]amino]-6-(4-hydroxy-6-methyl-2,3-dihydrobenzofuran-5-yl)-4-methyl-1,2,4-triazin-5-one

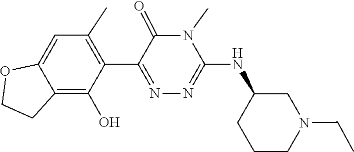 [SMILES: CCN1CCC[C@@H](Nc2nnc(-c3c(C)cc4c(c3O)CCO4)c(=O)n2C)C1]

### Step A: 6-(4-Benzyloxy-6-methyl-2,3-dihydrobenzofuran-5-yl)-4-methyl-3-methylsulfanyl-1,2,4-triazin-5-one

A mixture of 2-(4-benzyloxy-6-methyl-2,3-dihydrobenzofuran-5-yl)-4,4,5,5-tetramethyl-1,3,2-dioxaborolane (217.0 mg, 0.59 mmol, 1.12 eq, CAS #2923540-53-6), 6-iodo-4-methyl-3-methylsulfanyl-1,2,4-triazin-5-one Intermediate 2 (150.0 mg, 0.53 mmol, 1.0 eq), K₂CO₃ (220.0 mg, 1.59 mmol, 3.0 eq) and Pd-170 (71.0 mg, 0.11 mmol, 0.2 eq) in MeCN (10 mL) and water (2 mL) were degassed with N₂ for 5 mins, then was heated to 60° C. for 2 h. A slow stream of N₂ was added via a needle to the reaction mixture to try limit formation of diMeS side-products. The reaction was left to stir for ~4 days. The reaction was cooled, concentrated and loaded onto Celite. The crude product was purified by column chromatography on C18 silica gel (40 g cartridge, 10-100% [0.1% formic acid MeCN: 0.1% aq. formic acid], to afford the title compound (36.0 mg, 17% yield) as a yellow solid. LCMS m/z 395.8 [M+H]⁺ ESI pos.

### Step B: 6-(4-Benzyloxy-6-methyl-2,3-dihydrobenzofuran-5-yl)-4-methyl-3-[[(3R)-1-benzyl-3-piperidyl]amino]-1,2,4-triazin-5-one; formic acid salt

6-(4-Benzyloxy-6-methyl-2,3-dihydrobenzofuran-5-yl)-4-methyl-3-methylsulfanyl-1,2,4-triazin-5-one (35.0 mg, 0.09 mmol, 1.0 eq) was added to a small MW vial and (R)-3-amino-1-benzylpiperidine (160.0 mg, 0.84 mmol, 9.5 eq; CAS #168466-84-0) was added. The mixture was then irradiated at 180° C. for 1 h. The reaction was cooled to rt, the resulting thick black residue dissolved with DCM and loaded onto Celite. The crude product was purified by reverse-phase column chromatography on C18 silica gel (26 g cartridge, 10-100% [0.1% formic acid MeCN: 0.1% aq formic acid], to afford the title compound (30.0 mg, 54% yield) as a brown solid. LCMS m/z 538.4 [M+H]⁺ ESI pos.

### Step C: 3-[[(3R)-1-Ethyl-3-piperidyl]amino]-6-(4-hydroxy-6-methyl-2,3-dihydrobenzofuran-5-yl)-4-methyl-1,2,4-triazin-5-one

Pd/C (Type 39) (24.0 mg, 0.01 mmol, 0.26 eq) and Pd/C (Type 87) (12.0 mg, 0.01 mmol, 0.26 eq) were added to a stirred solution of 6-(4-benzyloxy-6-methyl-2,3-dihydrobenzofuran-5-yl)-4-methyl-3-[[(3R)-1-benzyl-3-piperidyl]amino]-1,2,4-triazin-5-one; formic acid salt (30.0 mg, 0.04 mmol, 1.0 eq) in acetonitrile (3.0 mL). The hydrogenation vessel was placed under an atmosphere of hydrogen gas (2 bar) at rt and vigorously stirred for 18 h. The reaction was filtered through a plug of celite, rinsing with EtOH, and concentrated to dryness to give the crude product (14 mg). The crude product was purified by column chromatography on silica gel (12 g cartridge, 0-10% MeOH (0.7 M NH₃)/DCM) to afford the title compound (8.0 mg, 47% yield) after freeze-drying as a light yellow freeze-dried solid. LCMS m/z 386.2 [M+H]⁺ ESI pos.

### Example 6

### 6-(4-Chloro-2-hydroxy-6-methyl-phenyl)-3-[[(3R)-1-ethyl-3-piperidyl]amino]-4-methyl-1,2,4-triazin-5-one

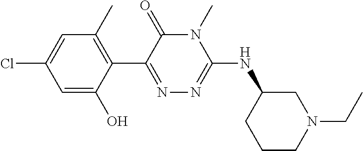 [SMILES: CCN1CCC[C@@H](Nc2nnc(-c3c(C)cc(Cl)cc3O)c(=O)n2C)C1]

### Step A: 6-(4-Chloro-2-hydroxy-6-methyl-phenyl)-4-methyl-3-methylsulfanyl-1,2,4-triazin-5-one

A mixture of (4-chloro-2-hydroxy-6-methyl-phenyl)boronic acid (200.0 mg, 1.07 mmol, 1.03 eq; CAS #1207961-50-9), 6-iodo-4-methyl-3-methylsulfanyl-1,2,4-triazin-5-one Intermediate 2 (295.0 mg, 1.04 mmol, 1.0 eq), K₂CO₃ (430.0 mg, 3.11 mmol, 2.99 eq) and Pd-170 (140.0 mg, 0.21 mmol, 0.2 eq) in MeCN (12 mL) and water (3 mL) were degassed with N₂ for 5 mins, then was heated to 60° C. for 20 h. The reaction was cooled, concentrated and loaded onto Celite. The crude product was purified by column chromatography on C18 silica gel (40 g cartridge, 10-100% [0.1% formic acid MeCN: 0.1% aq. formic acid], to give the title compound (132.0 mg, 39% yield) as a light yellow solid. LCMS m/z 298.1/300.1 [M+H]⁺ ESI pos.

### Step B: 6-(4-Chloro-2-hydroxy-6-methyl-phenyl)-3-[[(3R)-1-ethyl-3-piperidyl]amino]-4-methyl-1,2,4-triazin-5-one

A MW vial containing, 6-(4-chloro-2-hydroxy-6-methyl-phenyl)-4-methyl-3-methylsulfanyl-1,2,4-triazin-5-one (65.0 mg, 0.22 mmol, 1.0 eq) and (3R)-1-ethylpiperidin-3-amine (121.0 mg, 0.94 mmol, 4.32 eq) was irradiated at 180° C. for 0.5 h wherein nearly complete conversion was seen. The reaction mixture was concentrated to dryness then loaded onto Celite. The crude was then purified by reverse-phase flash column chromatography (C18 13 g cartridge, 10-100% MeCN/aq. 10 mM NH₄HCO₃) to afford the title compound (35.0 mg, 40% yield) as a light yellow solid. LCMS m/z 378.3/380.2 [M+H]⁺, ESI pos.

### Example A

A compound of formula I can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula I can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. A compound of formula I 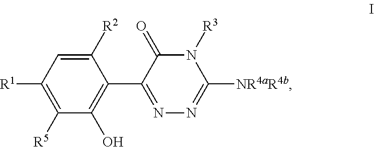
Wherein,
R¹ is H, acetyl, SF₅, halo, alkyl, alkoxy, haloalkyl, haloalkoxy or cyano; and R⁵ is H; or
R¹ and R⁵, and the atoms to which they are bonded, form either a 4- to 6-membered heterocycle ring comprising a single O heteroatom optionally substituted with one or two substituents, each independently selected from halo and alkyl, or R¹ and R⁵, and the atoms to which they are bonded, form a 4- to 6-membered cycloalkyl ring optionally substituted with one or two substituents independently selected from halo and alkyl;
R² is H, halo, alkyl, alkoxy, alkoxyalkyl, haloalkyl, or cycloalkyl, wherein cycloalkyl is optionally substituted with halo;
R³ is H or alkyl; and
R^{4a} is a heterocycle ring optionally substituted with one to three substituents, each independently selected from halo, alkyl, haloalkyl, hydroxyalkyl, —OH, oxo, —CO₂H, cycloalkylalkyl or cycloalkyl optionally substituted with halo, and R^{4b} is H, or
R^{4a} and R^{4b}, and the N to which they are bonded, form a 9-membered heterocycle comprising one or two N heteroatoms optionally substituted with one or two substituents, each independently selected from alkyl, —OH or halo;
and pharmaceutically acceptable salts thereof.

2. A compound according to claim 1, wherein
R¹ is halo, haloalkyl or haloalkoxy and R⁵ is H; or
R¹ and R⁵, and the atoms to which they are bonded, form either a 4- to 6-membered heterocycle ring comprising a single O heteroatom, each optionally substituted with one or two substituents, each independently selected from halo and alkyl, or
R¹ and R⁵, and the atoms to which they are bonded, form a 4- to 6-membered cycloalkyl ring optionally substituted with one or two substituents independently selected from halo and alkyl.

3. A compound according to claim 1, wherein
R¹ is halo, haloalkyl or haloalkoxy, and R⁵ is H; or
R¹ and R⁵ and the atoms to which they are bonded, form either a 5-membered heterocycle ring comprising a single O heteroatom, or
R¹ and R⁵, and the atoms to which they are bonded, form a 5-membered cycloalkyl ring.

4. A compound according to claim 1, wherein R¹ is halo, haloalkyl or haloalkoxy.

5. A compound according to claim 1, wherein R¹ is haloalkyl.

6. A compound according to claim 1, wherein
R⁵ is H, or
R¹ and R⁵, and the atoms to which they are bonded, form either a 5-membered heterocycle ring comprising a single O heteroatom, or
R¹ and R⁵, and the atoms to which they are bonded, form a 5-membered cycloalkyl ring.

7. A compound according to claim 1, wherein R⁵ is H.

8. A compound according to claim 1, wherein R² is H or alkyl.

9. A compound according to claim 1, wherein R² is H.

10. A compound according to claim 1, wherein R³ is alkyl.

11. A compound according to claim 1, wherein
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-membered heterocycle comprising one or two N heteroatoms optionally substituted with alkyl.

12. A compound according to claim 1, wherein
R^{4a} is a 6-membered heterocycle ring comprising a single N heteroatom substituted with alkyl, and R^{4b} is H, or
R^{4a} and R^{4b}, and the N to which they are bonded, form a 9-membered heterocycle comprising one or two N heteroatoms optionally substituted with alkyl.

13. A compound according to claim 1, wherein
R^{4a} is a 6-membered heterocycle ring comprising a single N heteroatom substituted with alkyl, and R^{4b} is H, or
R^{4a} and R^{4b}, and the N to which they are bonded, form a 9-membered heterocycle comprising two N heteroatoms wherein the 9-membered heterocycle comprising two N heteroatoms is substituted with alkyl.

14. A compound according to claim 1, wherein R^{4a} is ethylpiperidyl and R^{4b} is H.

15. A compound according to claim 1, wherein
R¹ is halo, haloalkyl, or haloalkoxy; and R⁵ is H; or
R¹ and R⁵, and the atoms to which they are bonded, form either a 5-membered heterocycle ring comprising a single O heteroatom, or R¹ and R⁵, and the atoms to which they are bonded, form a 5-membered cycloalkyl ring;
R² is H, halo, alkyl, haloalkyl, or cycloalkyl, wherein cycloalkyl is optionally substituted with halo;
R³ is alkyl; and
R^{4a} is a 6-membered heterocycle ring substitute with alkyl, hydroxyalkyl, cycloalkylalkyl or cycloalkyl optionally substituted with halo, and R^{4b} is H, or
R^{4a} and R^{4b}, and the N to which they are bonded, form a 9-membered heterocycle comprising one or two N heteroatoms optionally substituted with alkyl or halo;
and pharmaceutically acceptable salts thereof.

16. A compound according to claim 1, wherein
R¹ is halo, haloalkyl, or haloalkoxy; and R⁵ is H; or
R¹ and R⁵, and the atoms to which they are bonded, form either a 5-membered heterocycle ring comprising a single O heteroatom, or
R¹ and R⁵, and the atoms to which they are bonded, form a 5-membered cycloalkyl ring;
R² is H or alkyl;
R³ is alkyl; and
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-membered heterocycle comprising one or two N heteroatoms optionally substituted with alkyl;
and pharmaceutically acceptable salts thereof.

17. A compound according to claim 1, wherein
R¹ is halo, haloalkyl, or haloalkoxy; and R⁵ is H; or
R¹ and R⁵, and the atoms to which they are bonded, form either a 5-membered heterocycle ring comprising a single O heteroatom, or R¹ and R⁵, and the atoms to which they are bonded, form a 5-membered cycloalkyl ring;
R² is H or alkyl;
R³ is alkyl; and
R^{4a} is a 6-membered heterocycle ring comprising a single N heteroatom substituted with alkyl or hydroxyalkyl, and R^{4b} is H, or
R^{4a} and R^{4b}, and the N to which they are bonded, form a 9-membered heterocycle comprising two N heteroatoms wherein the 9-membered heterocycle comprising two N heteroatoms is substituted with alkyl;
and pharmaceutically acceptable salts thereof.

18. A compound according to claim 1, wherein
R¹ is halo, haloalkyl, or haloalkoxy; and R⁵ is H; or
R¹ and R⁵, and the atoms to which they are bonded, form either a 5-membered heterocycle ring comprising a single O heteroatom, or
R¹ and R⁵, and the atoms to which they are bonded, form a 5-membered cycloalkyl ring;
R² is H or alkyl;
R³ is alkyl; and
R^{4a} is a 6-membered heterocycle ring comprising a single N heteroatom substituted with alkyl, and R^{4b} is H, or
R^{4a} and R^{4b}, and the N to which they are bonded, form a 9-membered heterocycle comprising one or two N heteroatoms optionally substituted with alkyl;
and pharmaceutically acceptable salts thereof.

19. A compound according to claim 1, wherein
R¹ is halo, haloalkyl, or haloalkoxy; and R⁵ is H; or
R¹ and R⁵, and the atoms to which they are bonded, form either a 5-membered heterocycle ring comprising a single O heteroatom, or
R¹ and R⁵, and the atoms to which they are bonded, form a 5-membered cycloalkyl ring;
R² is H or alkyl;
R³ is alkyl; and
R^{4a} is a 6-membered heterocycle ring comprising a single N heteroatom substituted with alkyl, and R^{4b} is H, or
R^{4a} and R^{4b}, and the N to which they are bonded, form a 9-membered heterocycle comprising two N heteroatoms wherein the 9-membered heterocycle comprising two N heteroatoms is substituted with alkyl;
and pharmaceutically acceptable salts thereof.

20. A compound according to claim 1, wherein
R¹ is halo, haloalkyl, or haloalkoxy; and R⁵ is H; or
R¹ and R⁵, and the atoms to which they are bonded, form either a 5-membered heterocycle ring comprising a single O heteroatom, or
R¹ and R⁵, and the atoms to which they are bonded, form a 5-membered cycloalkyl ring;
R² is alkyl;
R³ is alkyl; and
R^{4a} is a 6-membered heterocycle ring comprising a single N heteroatom substituted with alkyl, and R^{4b} is H, or
R^{4a} and R^{4b}, and the N to which they are bonded, form a 9-membered heterocycle comprising two N heteroatoms wherein the 9-membered heterocycle comprising two N heteroatoms is substituted with alkyl;
and pharmaceutically acceptable salts thereof.

21. A compound according to claim 1, selected from
3-[[(3R)-1-Ethyl-3-piperidyl]amino]-6-[2-hydroxy-4-(trifluoromethyl)phenyl]-4-methyl-1,2,4-triazin-5-one; and
3-[[(3R)-1-ethyl-3-piperidyl]amino]-6-[2-hydroxy-6-methyl-4-(trifluoromethyl)phenyl]-4-methyl-1,2,4-triazin-5-one;
and pharmaceutically acceptable salts thereof.

22. A compound according to claim 1, selected from
3-[[(3R)-1-Ethyl-3-piperidyl]amino]-6-(4-hydroxy-6-methyl-indan-5-yl)-4-methyl-1,2,4-triazin-5-one;
3-[[(3R)-1-(2-Hydroxyethyl)-3-piperidyl]amino]-6-(4-hydroxy-6-methyl-indan-5-yl)-4-methyl-1,2,4-triazin-5-one;
3-[[(3R)-1-Ethyl-3-piperidyl]amino]-6-(4-hydroxy-6-methyl-2,3-dihydrobenzofuran-5-yl)-4-methyl-1,2,4-triazin-5-one; and
6-(4-Chloro-2-hydroxy-6-methyl-phenyl)-3-[[(3R)-1-ethyl-3-piperidyl]amino]-4-methyl-1,2,4-triazin-5-one;
and pharmaceutically acceptable salts thereof.

23. A compound according to claim 1, wherein the compound is 3-[[(3R)-1-ethyl-3-piperidyl]amino]-6-[2-hydroxy-6-methyl-4-(trifluoromethyl)phenyl]-4-methyl-1,2,4-triazin-5-one or pharmaceutically acceptable salts thereof.

24. A compound according to claim 1 wherein the compound is selected from
3-[[(3R)-1-Ethyl-3-piperidyl]amino]-6-(4-hydroxy-6-methyl-indan-5-yl)-4-methyl-1,2,4-triazin-5-one; and
3-[[(3R)-1-Ethyl-3-piperidyl]amino]-6-(4-hydroxy-6-methyl-2,3-dihydrobenzofuran-5-yl)-4-methyl-1,2,4-triazin-5-one;
and pharmaceutically acceptable salts thereof.

25. A process to prepare a compound according to claim 1, comprising reacting a compound of formula III to provide a compound of formula I through a protecting group (PG) cleavage

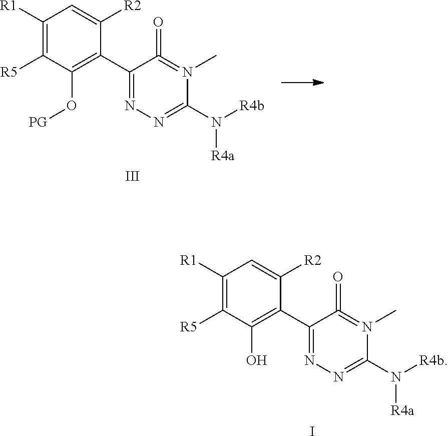

26-27. (canceled)

28. A pharmaceutical composition comprising a compound according to claim 1 and a therapeutically inert carrier.

29-32. (canceled)

33. A method of inhibiting NLRP3 in a subject in need thereof, which method comprises administering to the subject an effective amount of a compound NLRP3 according to claim 1.

34. A method for treatment of a disease, disorder or condition in a subject in need thereof which method comprises administering to the subject an effective amount of a compound according to claim 1, wherein the disease, disorder or condition is selected from Alzheimer's disease and Parkinson's disease.

35-36. (canceled)

37. A compound according to claim 1, wherein
R¹ is halo, haloalkyl or haloalkoxy, and R⁵ is H; or
R¹ and R⁵ and the atoms to which they are bonded, form either a 5-membered heterocycle ring comprising a single O heteroatom, or
R¹ and R⁵, and the atoms to which they are bonded, form a 5-membered cycloalkyl ring;
R² is H or alkyl;
R³ is alkyl;
R^{4a} is a 6-membered heterocycle ring comprising a single N heteroatom substituted with substituted with alkyl or hydroxyalkyl; and
R^{4b} is H.
